(19) United States
(12) Patent Application Publication
(10) Pub. No.: US 2018/0028703 A1
(43) Pub. Date: Feb. 1, 2018

(54) TRAY FOR HOLDING STERILIZATION TARGETS

(71) Applicant: rms Surgical, Inc., Elk River, MN (US)

(72) Inventors: Alexander McLaughlin, Isanti, MN (US); Doug McLane, Elk River, MN (US)

(21) Appl. No.: 15/220,757

(22) Filed: Jul. 27, 2016

(51) Int. Cl. A61L 2/26; B65D 43/02; B65D 1/38; B65D 25/28; A61B 50/33; B65D 1/34

(52) U.S. Cl. CPC A61L 2/26; A61B 50/33; B65D 1/34; B65D 1/38; B65D 25/2841; B65D 43/02; A61L 2202/24

(57) ABSTRACT

Disclosed embodiments include a tray configured for holding various sterilization targets. The tray may include a perforated floor member connected to a plurality of vertically oriented walls, which may also be perforated. Together, the floor member and walls define an internal cavity that includes at least one wire support for removably holding at least one sterilization target, such as a medical instrument and/or implant or components thereof, within the internal cavity. Each wire support may include two end inserts and a middle portion, the two inserts secured via connection means to one or more walls of the tray. The connection means may comprise mounting fixtures that further include a plurality of receiving slots or holes each configured to receive an insert. The wire supports may increase sterilant flow and circulation, thereby improving sterilization of instruments and/or implants held within the tray disclosed herein.

### TECHNICAL FIELD

The present application relates to trays for holding various sterilization targets. More particularly, the present application provides perforated trays with at least one wire support for removably holding at least one medical instrument and/or implant and/or other sterilizable component during sterilization and methods for making the wire supports. The trays include connection means for securing the wire supports to the tray.

### BACKGROUND

Medical instruments and/or implants often come in a wide range of diverse shapes and sizes, each being customized to perform highly specialized tasks with high precision. For ease of use and organization, assorted instruments are often packaged together in procedure-specific collections or kits, which are housed in various containers. Constructing these containers with the durability necessary to protect expensive medical instrumentation from damage is imperative, and the variety of stresses applied to such containers makes this a challenging task. For example, containers used to provide instruments for use on patients must be able to withstand the high heat and pressure typically associated with autoclave sterilization. The same containers may also be relied upon to house the instruments during shipping and storage. Throughout each of these processes, the instruments must be further shielded from the damage caused by instrument-to-instrument contact without diminishing the total holding capacity of each container.

In addition to protecting the instruments from damage, the containers must organize the instruments in a way that ensures their thorough sterilization. Specifically, the containers must arrange the instruments in a manner that maximizes their exposure to the steam and/or chemicals employed for sterilization. Internal structures for holding or propping variously-shaped instruments may be included for this purpose.

Current containers fail to successfully perform the aforementioned functions, especially with respect to instrument sterilization, and/or are expensive to manufacture. In particular, preexisting containers may comprise sterilization trays with brackets, posts, cut-out blocks, or fixtures to organize various instruments, but the materials and design of these internal features typically reduce or limit sterilant exposure to the instruments and disrupt overall sterilant flow and circulation. Some container designs are susceptible to breaking or cracking during transit or sterilization. Thus, improved trays for medical instruments and/or implants are needed to avoid the harmful consequences of incomplete sterilization and ensure safe storage and transport of expensive medical equipment.

### SUMMARY

A tray for holding sterilization targets is described that comprises: a perforated floor member; a plurality of vertically oriented walls connected to the floor member around a perimeter of the floor member such as to define an internal cavity, wherein one or more of the walls may be perforated; at least one wire support for removably holding and supporting against lateral or longitudinal movement at least one sterilization target within the internal cavity, wherein each wire support comprises two ends and a middle portion, the middle portion comprising at least two bends in a wire from which the support is made; and connection means associated with at least one of the plurality of vertically oriented walls for securing each end of the at least one wire support to the tray.

A method of forming a wire support for use in a tray for holding sterilization targets comprising: (i) a perforated floor member; and (ii) a plurality of vertically oriented walls connected to the floor member around a perimeter of the floor member, wherein one or more of the walls may be perforated and the floor member and walls together define a sterilization space enclosed by the tray is also described. The method comprising: using a computer aided design tool programmed with a dimensioned design description for the tray for holding sterilization targets, including the sterilization space, defining a location in the sterilization space for at least one sterilization target; using a computer aided design tool programmed with a dimensioned design description for the at least one sterilization target, identifying at least one contact point on the sterilization target, for supporting or positioning the sterilization target at its location in the sterilization space; using a computer aided design tool programmed with the bending capabilities of a wire bending machine and a wire specification, defining a wire path between two connection means associated with at least one of the plurality of vertically oriented walls for securing each end of the at least one wire support to the tray, said wire path defining a wire configuration extending between two end points of at least one wire support segment and passing through the at least one contact point and at least one wire connection segment at each end of the wire support segment defined by a connecting bend in the wire path; storing in a storage medium a wire path design file including the location of a plurality of end points of the support segments and connection segments and a radius or other specification of the connecting bends, said wire path forming a continuous path between the two connection means; transmitting to the control unit of the wire bending machine the wire path design file; and causing the wire bending machine to perform bending of a wire supplied to the bending machine as defined by the wire path design file, including forming an insert for each of the connection means by bending and cutting wire at each end of the wire path.

### DESCRIPTION OF THE DRAWINGS

In the drawings:

FIG. 1 is an isometric view of a tray and corresponding cover piece, showing various medical instruments suspended upon wire supports within the tray, each wire support secured to mounting fixtures along the tray perimeter.

FIG. 2 is a side view of the tray of FIG. 1 with one side removed and with various instruments suspended therein on wire supports configured for the set of instruments shown.

FIG. 3 is a magnified side view of the tray of FIG. 1 showing a plurality of perforations throughout a side wall of the tray and the outer edge of a mounting fixture supported on the side wall.

FIG. 4 is another isometric view of the tray of FIG. 1, further showing a caddy for holding smaller medical instruments and/or implant components.

FIG. 5 is a top view of a tray as in FIG. 1 in another embodiment, showing a different set of medical instruments and wire supports within the tray, including a caddy attached to one of the mounting fixtures.

FIG. 6 is a top view of the tray of FIG. 1.

FIG. 7 is an isometric view of the tray of FIG. 1 with one side wall removed, revealing a cross-sectional view of a mounting fixture attached to the removed side wall and used to removably secure wire supports within the tray.

FIG. 8 is another isometric view of the tray, showing a set of supports formed from wire and particularly configured for a specific set of instruments.

FIG. 9 is a magnified isometric top view of the tray and two supports formed from wire near one end of the tray.

FIG. 10 is a magnified isometric top view of the tray and supports formed from wire near the longitudinal middle of the tray.

FIG. 11 is a magnified isometric top view of the tray and supports formed from wire near a second end of the tray.

FIG. 12 is a cross-sectional view of the tray in one embodiment, showing a bent top edge end connector with two aligned holes securing one end of a wire support to the tray.

FIG. 13 is magnified isometric top view of the tray of FIG. 7, without wire supports, showing the locations of a plurality of medical instruments in a sterilization space defined by the tray.

FIG. 14 is magnified isometric top view of the plurality of medical instruments as located in the sterilization space defined by a tray (which is not shown) and showing a wire path for the leftmost wire support in FIG. 7, as defined by the location in the sterilization space defined by the tray of end points of support segments, connection segments and bends.

FIG. 15 is magnified isometric top view of the plurality of medical instruments FIG. 14 as located in the sterilization cavity defined by tray and showing the leftmost wire support in FIG. 7, as formed by a wire bending machine programmed with the wire path of FIG. 14.

FIG. 16 shows a flowchart describing major steps in an exemplary wire design and forming process.

### DETAILED DESCRIPTION

### Overview

Provided herein are improved trays for sterilizing, shipping, and storing various sterilization targets. For instance, the trays disclosed herein comprise one or more wire supports customized to removably hold specific medical instruments and/or implants or components thereof during sterilization processes, when they become sterilization targets. The wire supports are configured to increase sterilant flow, sterilant exposure, and the instrument-holding capacity of the trays while enhancing overall durability. The trays may also comprise perforated walls to further enhance the flow of sterilants, and connection means for securing variously-configured wire supports within each tray.

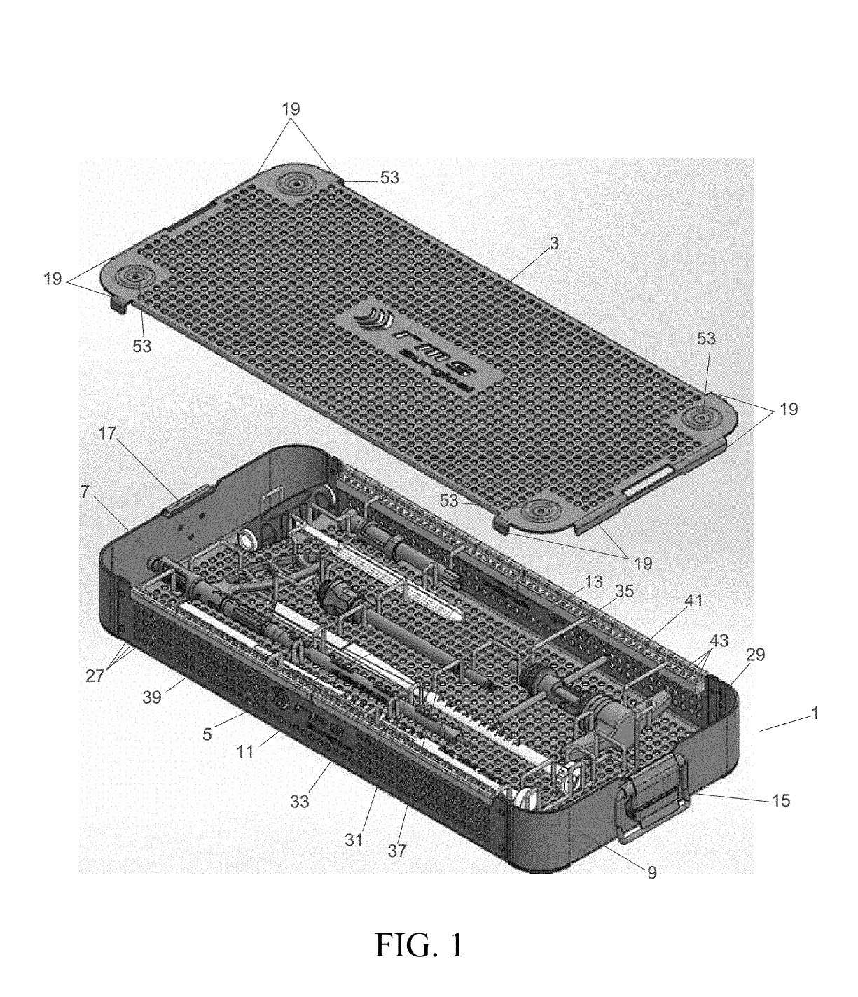

Referring to the drawings, FIG. 1 shows a tray 1 with a corresponding cover 3. In the particular embodiment shown, tray 1 comprises a floor member 5, two vertically oriented end walls 7 and 9, and two vertically oriented side walls 11 and 13 connected together to define an interior space or cavity 29. Within cavity 29, one or more wire supports 31 are anchored to tray 1 via connection means, such as receiving slots or holes 43 arranged within mounting fixtures 39 and 41, as depicted. Such receiving slots or holes 43 may be configured to slidably receive the inserts 33, 35 that comprise the end portions of each wire support 31. Tray 1 also includes a plurality of perforations 27 for improving sterilant flow and circulation, which may be included in floor member 5, cover 3, one or more side walls 11 and 13, and/or one or more end walls 7 and 9. In the exemplary embodiment shown in FIG. 1, perforations 27 are included throughout side walls 11 and 13, floor member 5, and cover 3. Tray 1 may also comprise two handle assemblies 15 and 17 positioned on an exterior surface of end walls 7 and 9, for example, which may contribute to the attachment of cover 3 to tray 1.

### Tray

In the particular configuration shown in FIG. 1, tray 1 comprises an approximately rectangular shape defined by floor member 5, end walls 7 and 9, and side walls 11 and 13. In embodiments not shown, tray 1 may comprise a variety of other shapes. For example, tray 1 may be circular, triangular, square, cylindrical, or irregular in shape. The shape of tray 1 may be adjusted depending on the sterilization targets contained therein, the interior confines of various sterilization devices, and/or the desired stacking capabilities of tray 1. Accordingly, the length, width, and/or height of tray 1 may also vary. In embodiments, the length of tray 1 may range from about 6 to about 48 inches, about 12 to about 36 inches, about 18 to about 30 inches, or about 22 to about 26 inches. The width of tray 1 may range from about 3 to about 48 inches, about 8 to about 36 inches, about 12 to about 24 inches, or about 15 to about 21 inches. The height of tray 1 may range from about 0.5 to about 12 inches, about 1 to about 9 inches, about 2 to about 8 inches, about 3 to about 6 inches, or about 4 to about 5 inches.

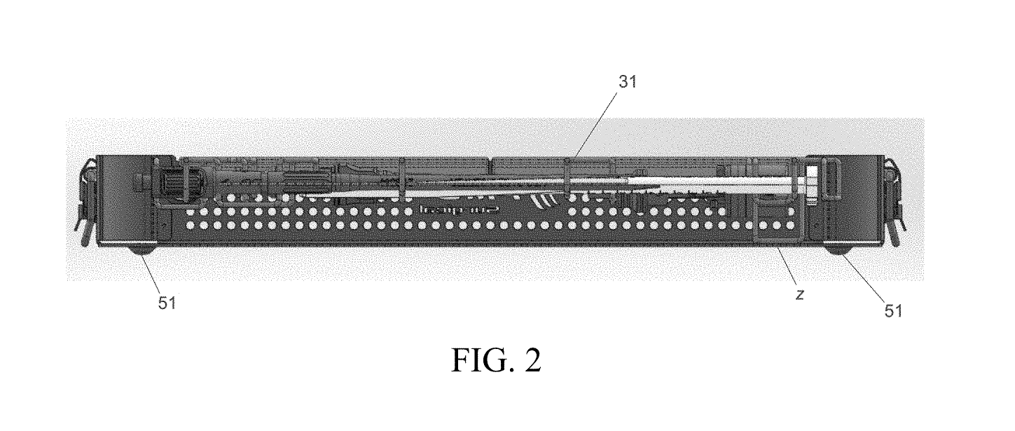

As shown in FIG. 2, tray 1 may also comprise one or more embossed feet or legs 51 positioned beneath floor member 5. Legs 51 may comprise various lengths, may protrude at various angles, and may be positioned at various locations beneath floor member 5. In preferred embodiments, legs 51 are configured such that tray 1 remains parallel to its resting surface. In other embodiments, legs 51 may each comprise different lengths, or extend at different angles relative to floor member 5, to prop tray 1 at an angle, thereby facilitating drainage of liquid materials from tray 1 after sterilization and/or cleaning. Legs 51 may align with corresponding depressed areas 53 within cover 3 to facilitate stacking of multiple trays. In embodiments, legs 51 may create a gap between vertically stacked trays 1, allowing for sterilant flow and circulation between stacked trays and improving sterilant movement through floor member 5 and cover 3 of each tray 1. In additional embodiments, tray 1 may further comprise various flanges on an exterior surface of cover 3 and/or floor member 5 to further facilitate the proper alignment and stability of trays 1 stacked in vertical columns.

As further shown in FIG. 1, cover 3 may include skirt portions 19 at or near each corner to position cover 3 and releasably secure cover 3 to tray 1. Skirt portions 19 may encompass various distances around the perimeter of cover 3. In some embodiments, the entire perimeter of cover 3 may be skirted, while in others, cover 3 may lack skirt portions 19 altogether. Skirt portions 19 may align with corresponding lips on the side and/or end walls of tray 1. Together, the skirt portions 19 and lips may secure cover 3 to tray 1 via a snapping mechanism or friction fit. In other designs, cover 3 may be coupled to tray 1 via hinged or removable attachment, so that cover 3 is rotatable or positionable between open and closed states and configurations. In additional embodiments, cover 3 may be slidably attached to tray 1 by side tracks that mate with skirt portions 19. According to such embodiments, tray 1 may include one or more tracks (not shown) for slidably receiving cover 3. Cover 3 may also comprise one or more depressed areas 53 to facilitate stacking of multiple trays by receiving inversely oriented legs or protrusions that extend downward from floor member 5 in trays stacked above the tray shown in FIG. 1.

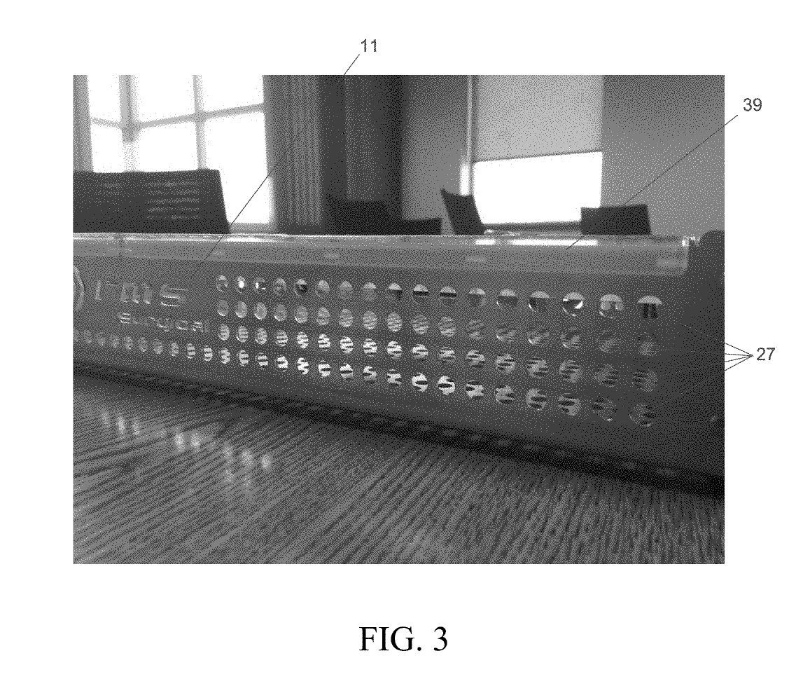

FIG. 3 is a magnified side view of tray 1, showing an external surface of side wall 11 or 13. As shown, a portion of each mounting fixture 39 or 41 may extend over the top of each side wall, covering a portion of the external surface of the side wall. The side wall may further comprise a plurality of perforations 27.

Perforations 27 may enhance the circulation and flow of various sterilants, allowing the sterilants to cover all surfaces of the sterilization targets, such as medical instruments and/or implants or components thereof, held within tray 1. Perforations 27 may be organized in various arrangements to direct the flow of sterilants in particular directions during a given sterilization process. For example, perforations 27 may be limited to opposing surfaces, e.g., side walls 11 and 13, to direct sterilant flow in one direction through tray 1. Perforations 27 may also be positioned to better accommodate different arrangements of multiple trays, e.g., vertical stacking, within a given sterilization chamber. For instance, perforations 27 may be positioned exclusively on cover 3 and floor member 5 to direct sterilant flow vertically through the top and bottom of each tray 1 stacked in an upright column. Such arrangements may also improve sterilant drainage at the conclusion of a sterilization process. Alternatively, perforations 27 may be included on each surface of tray 1 and cover 3 to maximize sterilant access to cavity 29. In addition to improving sterilant circulation and flow, perforations 27 may expedite cooling after sterilization by aerating tray 1. Furthermore, an increased number of perforations 27 may decrease the total weight of tray 1, making the tray easier to lift and handle.

The shape of each of the perforations 27 may vary. Perforations 27 shown in FIG. 3, for example, are each circular and uniform. In other embodiments, perforations 27 may each be square, rectangular, oval, elliptical, triangular, or T-shaped. Perforations 27 may also comprise slits of varying thickness. Perforations 27 present on a single tray may be uniformly shaped or differently shaped. For example, some of the perforations may be circular, while some of the perforations may comprise slits.

The density and/or total number of perforations 27 may also vary. For example, perforations 27 may be more or less numerous on various surfaces of tray 1 and/or cover 3. The density of perforations 27 may also vary across individual surfaces, for example, increasing in density near the longitudinal middle of each of the side and/or end walls. In other embodiments, the density may increase near the intersection between each of the side and end walls, i.e., at the corners of tray 1. In the particular embodiment shown in FIG. 3, perforations 27 are spaced uniformly with respect to each other. As shown also in FIGS. 1 and 3, the side walls 11, 13 may feature equally-spaced columns of perforations 27, with four perforations in each column. Floor member 5 and cover 3 may be substantially covered in perforations 27, except on the surfaces proximal to depressed areas 53 and legs 51, for example. Among the factors impacting the total number of perforations 27 on a single tray 1 include the size of the individual perforations, the size of the tray, the type of sterilization processes envisioned for a particular tray, and/or the user preferences for directional sterilant flow. In some embodiments, for example, the total number of perforations may range from about 1 to about 5000, about 500 to about 4000, about 1000 to about 3000, about 1500 to about 2500, about 1750 to about 2250, or about 1900 to about 2100 perforations. Per square inch, the number of perforations on any given surface of tray 1 may range from less than 1 to about 10, 15 or 20 perforations. Per square foot, the number of perforations on any given surface of tray 1 may range from less than 1 to about 100, 200 or 300 perforations.

The cross-sectional size of each individual perforation may also vary. In some embodiments, the cross-sectional width or diameter of each perforation may range from about 0.1 to about 5.0 cm, about 0.1 to about 3.0 cm, about 0.3 to about 2.5 cm, about 0.5 to about 2.0 cm, or about 1.0 to about 1.5 cm.

In alternative embodiments, some or all of perforations 27 may be replaced with one or more large openings. Such openings may be sized such that tray 1 comprises a box-like frame with visibly exposed wire supports 31 therein. In addition or alternatively, perforations 27 may be shaped to correspond to the size and/or shape of the particular instruments and/or implants held within cavity 29. For instance, an elongated, elliptical perforation may be positioned adjacent to a similarly elongated, elliptical instrument feature.

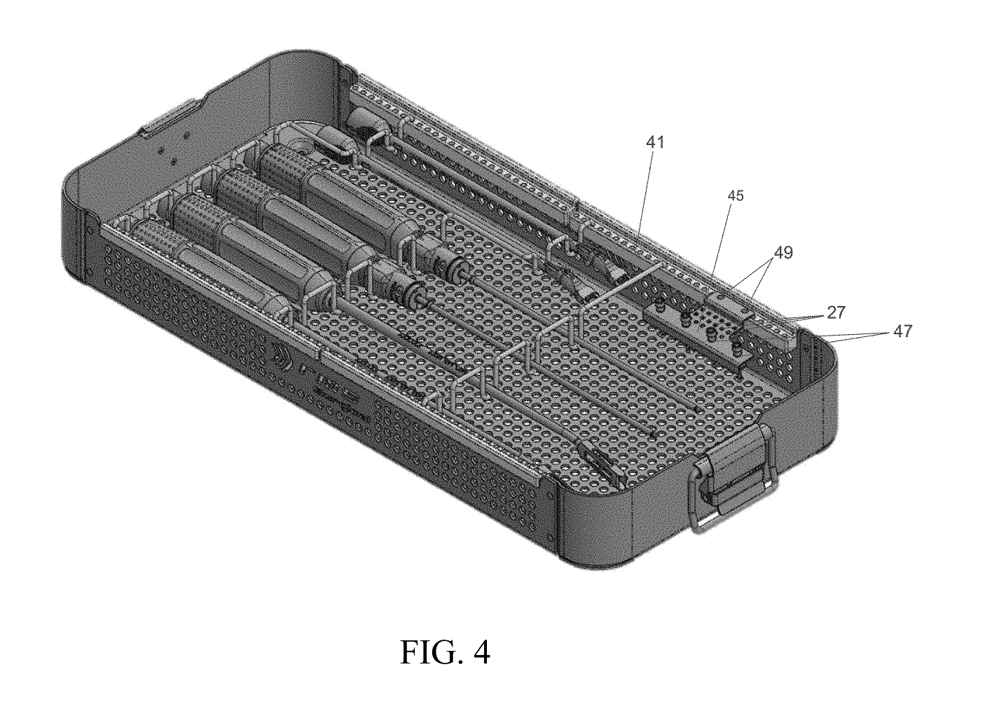

FIG. 4 is another isometric view of tray 1. In this particular embodiment, tray 1 includes a caddy 45 configured to releasably engage mounting fixtures 39 and/or 41. As such, caddy 45 may include at least one peg or protrusion 49 equal in width and shape to inserts 33 and 35 of each wire support 31, and thus similarly configured for slidable insertion into one or more receiving slots or holes 43. The caddy 45 may further comprise one or more receiving areas 47, e.g., holes or cavities, for holding narrower, shorter, and/or generally smaller medical instruments and/or implant components, e.g., screws, blades, scalpels, picks, or probes. One or more receiving areas 47 may be configured to contact each instrument at one or more positions, suspending each instrument a certain distance above floor member 5. In additional embodiments, caddy 45 may include a basket-like configuration or perforated indentations for receiving and holding multiple small instruments and/or implant components. Each caddy 45 may hold about 100 or more small instruments and/or implant components. A plurality of perforations 27 may also be included within caddy 45 to maximize sterilant exposure to the instruments held therein and to prevent caddy 45 from disrupting sterilant flow throughout internal cavity 29. Caddy 45, as shown in FIG. 4, is secured exclusively to mounting fixture 41, however, in additional embodiments caddy 45 may span the entire width of tray 1, releasably secured to mounting fixtures 39 and 41, for example.

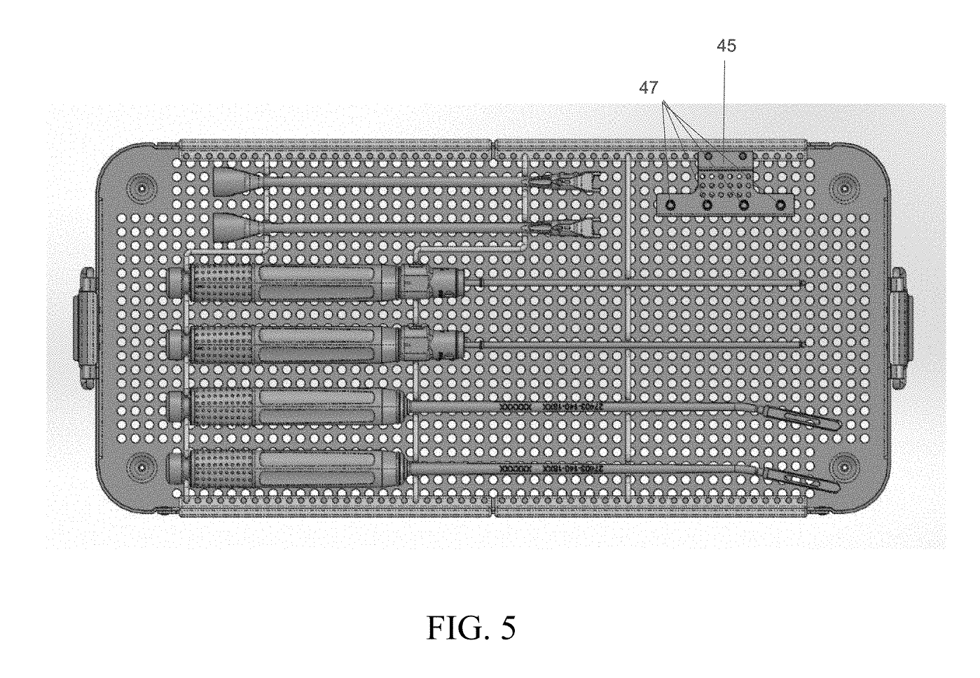

FIG. 5 is a top view of an embodiment of tray 1, which includes caddy 45 holding four small sterilization targets. In embodiments, tray 1 may hold a variety of elongated sterilization targets on one or more wire supports 31, and a variety of smaller sterilization targets on caddy 45. The particular arrangement of elongated sterilization targets shown in FIG. 5 ensures that such targets avoid interference with caddy 45. In embodiments, tray 1 may hold a number of elongated sterilization targets via one or more wire supports 31, the number ranging from about 1 to about 20, about 1 to about 15, about 1 to about 10, or about 1 to about 5. In embodiments, the number of sterilization targets supported via one or more wire supports 31 and caddy 45 may range from about 1 to about 200, about 1 to about 150, about 1 to about 100, about 1 to about 50, or about 1 to about 25.

Tray 1 may be used in a variety of sterilization processes. Such processes may include high pressure sterilization, high heat sterilization, gas sterilization, and/or heated or cold chemical sterilization. Accordingly, tray 1 may withstand a variety of conditions and/or devices, e.g., autoclaves. Temperatures during sterilization may reach up to or exceed about 400° F. and pressures may reach up to or exceed about 50 psi above atmospheric pressure. Sterilants employed pursuant to various sterilization processes may include but are not limited to: steam, water, gas, ethylene oxide gas, glutaraldehyde, formaldehyde, alcohol, and/or hydrogen peroxide. Commercial sterilant formulas may include products such as Cidex®, Clidox®, and/or Alcide®.

A wide variety of medical instruments and/or implants may be stored, reprocessed, transported, or sterilized within tray 1. Among the many examples compatible with tray 1 include, but are not limited to: various cutting devices, drilling devices, clamping devices, and/or depressors. Instruments may be grouped according to specific medical protocols such that one tray 1 is configured to hold a particular set of instruments and/or implants required for a given surgery, for example. Medical instruments may be further arranged according to the order and/or frequency with which they are needed during a specific medical procedure.

### Connection Means

Tray 1 may further include connection means for anchoring wire supports 31 to tray 1. Connection means may comprise various structural components, end connectors, connector pieces, or receptacles removably attached to, permanently affixed to, or integrally formed within tray 1. In the exemplary embodiments depicted in FIGS. 1-2 and 4-11, the connection means comprise mounting fixtures 39 and 41.

As shown particularly in FIG. 1, mounting fixtures 39 and 41 may be positioned along one or both side walls 11, 13 to removably secure wire supports 31 to tray 1. In addition or alternatively, mounting fixtures 39, 41 may be included on one or both end walls 7, 9, and/or floor member 5.

Each mounting fixture 39, 41 may comprise a plurality of receiving slots or holes 43, each slot or hole 43 with a defined depth configured to receive one insert 33 or 35 of a wire support 31. A typical depth for each slot or hole 43 may range from about 0.1 to about 2 inches. A cylindrical, vertical through-hole is used in one embodiment; such a through-hole receives a suitably formed insert 33, 35 of a wire support 31 and does not provide a “cup” in which sterilant fluid or debris may accumulate. By slidably inserting each insert 33, 35 into a different receiving slot or hole 43, the inserts of each wire support 31 may be moved to various positions along the length of tray 1. Thus, each wire support 31 may be re-positioned as desired by a user to accommodate different instruments and/or implants. Wire supports 31 may be repositioned for storage and handling. For instance, numerous wire supports 31 may be stored in tray 1 during periods of non-use to avoid losing wire supports 31 and protect them from damage.

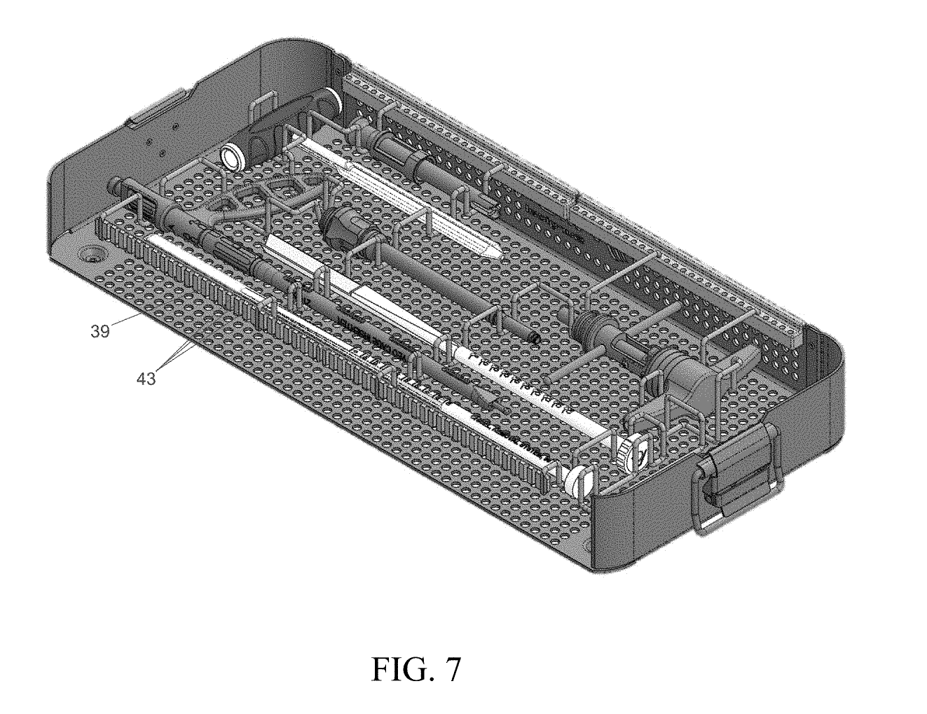

While in one embodiment a wire support 31 may be supported by inserts 33, 35 that are inserted at slots or holes 43 directly opposite each other, the configuration of a wire support 31 may lead to inserts 33, 35 that do not land in slots or holes 43 directly opposite each other; rather one insert 33 or 35 in a mounting fixture 39 or 41 may be displaced along the length of an opposing mounting fixture 39 or 41. Also, the configuration of a wire support 31 may, for example, lead to one insert 33, 35 inserted in a receiving slot or hole 43 of a mounting fixture 39, 41 on a side wall 11, 13, or one receiving slot or hole 43 on a side wall 11, 13 and one slot or hole 43 in a mounting fixture 39, 41 on an end wall 7, 9. Additionally, both inserts 33 and 35 may be inserted into different receiving slots or holes 43 on the same mounting fixture 39 or 41. The linear array of receiving slots or holes 43 of a mounting fixture 39 or 41, as shown in FIG. 7, allows end support for virtually any wire support 31 that may be designed to extend between side walls 11, 13 at any location along the length of mounting fixture 39 or 41.

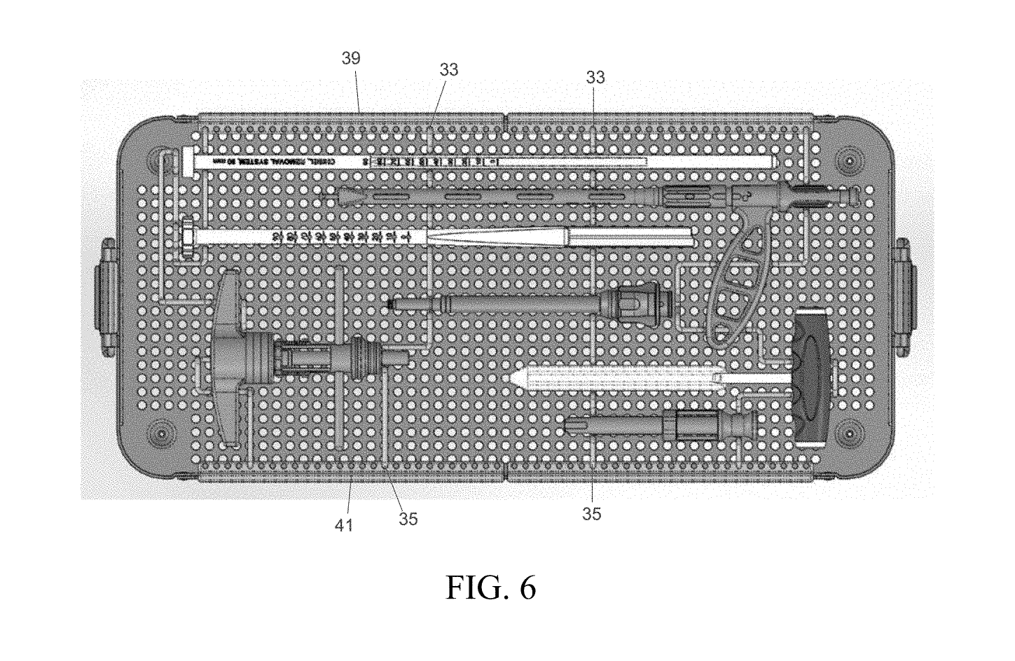

The number of receiving slots or holes 43 included in each mounting fixture 39 and/or 41 may vary. As shown in FIG. 6, for instance, mounting fixtures 39 and 41, each included in the same tray 1, may comprise an equal number of receiving slots or holes 43. In other embodiments, mounting fixtures 39 and 41 may each contain a different number of receiving slots or holes 43. Multiple mounting fixtures may be positioned adjacent to each other on the same side wall of tray 1. FIG. 6, for example, shows two mounting fixtures on each side wall 11, 13. The number of mounting fixtures arranged on one wall of tray 1 may be increased for larger trays and decreased for smaller trays. In some embodiments, the number of receiving slots or holes 43 in each mounting fixture may range from about 1 to about 100 receiving slots, about 2 to about 75 receiving slots, about 5 to about 60 receiving slots, about 5 to about 50 receiving slots, about 10 to about 40 receiving slots, about 15 to about 35 receiving slots, about 20 to about 30 receiving slots, or about 25 to about 30 receiving slots. Per linear inch, the number of receiving slots or holes 43 on any portion of each mounting fixture may range from less than about 1 to about 5 receiving slots or holes. Per linear foot, the number of receiving slots or holes 43 on any portion of each mounting fixture may range from less than about 1 to about 60 receiving slots or holes.

FIG. 7 depicts a cross-sectional profile of mounting fixture 39, made visible by removing side wall 11 from view. As shown, mounting fixture 39 comprises a plurality of receiving slots or holes 43, each slot or hole 43 having a vertical depth for slidably receiving one insert 33 or 35 of a wire support 31. Each insert 33, 35, once fully inserted into a receiving slot or hole 43, may span the entire depth of a receiving slot or hole 43, and may extend beyond the bottom of each slot or hole 43 such that the end point of each insert is visible beneath each receiving slot or hole 43 when viewed from a side view. In other examples, each insert 33, 35 may span only a fraction of the entire depth of each receiving slot or hole 43. The depth of insertion may be selected to provide the desired degree of stable holding and resistance to unintended removal of each insert 33, 35 from its slot or hole. For instance, the inserts 33, 35 may only span the depth of each receiving slot or hole 43 necessary to firmly secure each of the wire supports 31, and any objects resting thereon, to tray 1 without becoming detached or loose. The depth of receiving slots or holes 43 and/or the length of each insert 33, 35 may vary. In some embodiments, the depth of the receiving slots or holes 43 may range from about 0.1 to about 2.0 inches, about 0.1 to about 1.0 inch, about 0.2 to about 0.6 inches, or about 0.3 to about 0.5 inches.

Receiving slots or holes 43 may be compatible with variously shaped wire supports 31. As such, the shape and diameter defined by each of the receiving slots or holes 43 may correspond precisely to the similarly shaped inserts 33, 35 of wire supports 31. For instance, as shown in FIG. 6, receiving slots or holes 43 may be cylindrical to receive wire supports 31 with corresponding circular cross sections. Alternatively, to reduce areas where sterilization access is blocked or impaired, a receiving slot or hole 43 may be square or triangular and receive a wire of circular cross-section. In such embodiments, the gaps between an exterior surface of the inserts 33, 35 and an interior surface of the receiving slots or holes 43 may not interfere with the secure engagement of wire supports 31 with mounting fixtures 39, 41. Accordingly, mounting fixtures 39, 41 may be universally compatible with variously sized and/or shaped trays 1, each tray configured to receive interchangeable wire supports 31 with diverse structures. By comprising universal mounting fixtures, each compatible with a variety of wire supports 31, each tray 1 may be used for shipping, sterilizing, and storing a wide range of medical instruments, implants, and/or objects.

In other embodiments, the cross-sectional shape and/or size of wire supports 31 may vary, necessitating receiving slots or holes 43 of various shapes and/or sizes. For example, larger trays may include thicker wire supports 31, requiring receiving slots or holes 43 of greater width. Likewise, smaller trays 1 may comprise narrower receiving slots or holes 43 to more tightly receive correspondingly narrow inserts 33, 35. In other embodiments, receiving slots or holes 43 may comprise various additional shapes, e.g., square, rectangular, hexagonal, triangular, and/or irregular, to accommodate the cross-sectional designs of various wire supports 31 and/or maintain sterilant access to the interior of receiving slots or holes 43 and the inserts 33, 35 secured therein.

In additional embodiments, the diameter of each of the receiving slots or holes 43 may be adjusted by various means. For example, in one embodiment, receiving slots or holes 43 may each comprise an expandable portion or joint. Such expansion points may be manually adjusted by a user via a rotatable knob, for example. Other embodiments may comprise additional or alternative adjustment means. By enabling size adjustment of receiving slots or holes 43, individual mounting fixtures may be compatible with wire supports 31 of varying cross-sectional thickness and/or shape.

In addition or alternatively, inserts 33 and/or 35 of each wire support 31 may comprise spring-loaded mechanisms, e.g., latches or pins, for locking such inserts in place upon insertion into each receiving slot or hole 43 of mounting fixture 39, 41, or other end connector structures. According to such embodiments, a laterally-outward extending, spring-loaded latch or pin on each insert 33, 35 may compress upon insertion into a receiving slot or hole 43, remaining in a compressed state until each insert is fully inserted into the receiving slot or hole 43, at which point the spring-loaded latch or pin emerges through the bottom of the receiving slot or hole 43, springing laterally outward to prevent the insert from sliding upward through the receiving slot or hole 43 and thereby securing each wire support 31 in place. Such embodiments may be particularly necessary for receiving slots or holes of lesser depth, or horizontally-oriented receiving slots or holes unable to rely on gravity for securing inserts therein. Receiving slots or holes may comprise additional features for receiving such spring-loaded mechanisms, including lateral voids or cut-outs within each slot or hole 43 that correspond in size and shape to the latch or pin located on each insert. Upon proper alignment and full insertion of each insert into a receiving slot or hole 43, the void or cut-out receives the laterally-extending latch or pin and secures it in place until released by a user, for example.

Connection means, such as mounting fixtures 39, 41, may be permanently fixed to tray 1. In one embodiment, the connection means may comprise lock washers over-molded into side walls 11, 13. Alternatively, the connection means may be removably attached to tray 1. This may facilitate cleaning of a long contact surface between the structural pieces of the connection means and the associated vertically-oriented walls to which they are attached, which may be insufficiently exposed during sterilization. According to such embodiments, the connection means may be attached by various means. For example, mounting fixtures 39, 41 may be snapped onto the side and/or end walls of tray 1. In embodiments, various screws, adhesives, or locking mechanisms may be used to attach various connection means, including mounting fixtures 39, 41 and/or other end connectors, to tray 1.

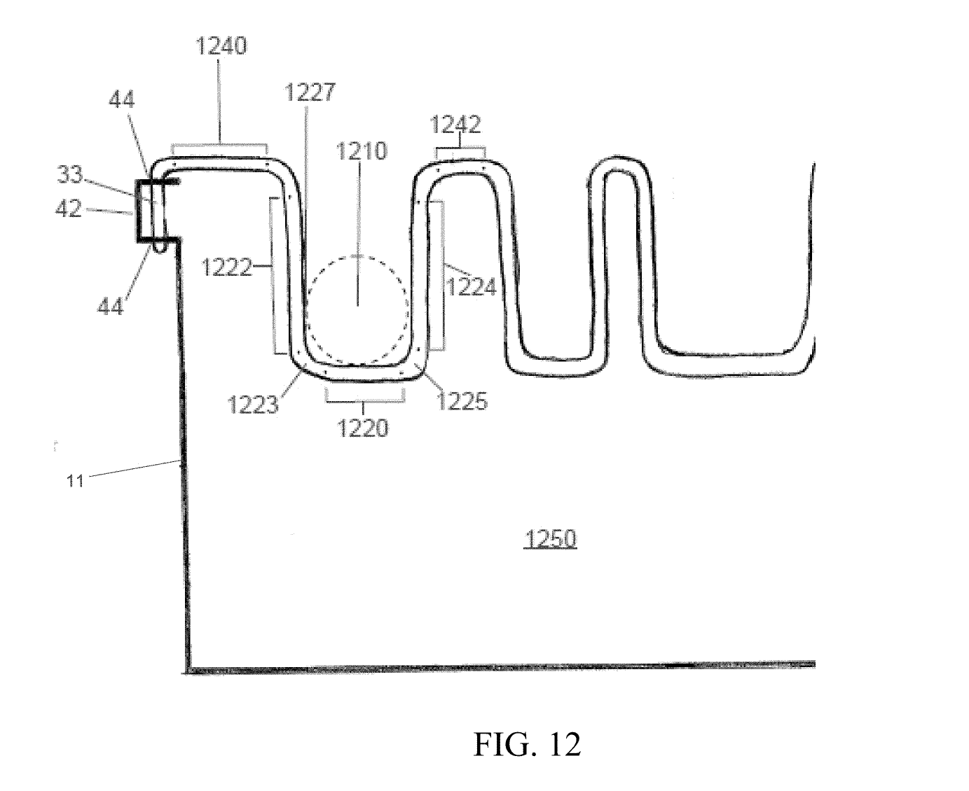

In additional embodiments, the connection means may comprise variously-structured end connectors integrally formed within tray 1. As shown in the cross-sectional view of tray 1 in FIG. 12, for example, one or more of the vertically oriented walls of tray 1 may comprise a bent top edge 42 with perforations or holes 44 for receiving inserts 33 and 35. The end connector defined by bent top edge 42 may comprise an approximately U-shaped protrusion that extends laterally outward at an approximately 90° angle with respect to the vertically oriented wall from which it protrudes. As shown, bent top edge 42 may further comprise one or more pairs of perforations or holes 44 vertically aligned to receive an end insert 33 or 35 of a wire support 31. Inserts 33, 35 may thus extend through both perforations or holes 44 to maintain the stability of the wire supports 31 attached thereto. Such pairs of perforations or holes 44 may extend along the entire length of bent top edge 42, or any portion thereof. Bent top edge 42 may additionally extend along the entire length of one or more vertically oriented walls of tray 1, or any portions thereof. In addition or alternatively, bent top edge 42 may comprise a plurality of perforations or holes 44 in only the top surface of bent top edge 42. In yet another embodiment not shown, bent top edge 42 may comprise only one laterally-outward extending surface, such that together with a side wall 11 or 13, bent top edge 42 forms an inverted L-shape when viewed from a cross-sectional side view. According to such an embodiment, bent top edge 42 comprises a perforated platform for receiving the inserts 33, 35. Another variation of bent top edge 42 may comprise a hollow, box-like end connector formed at the top of one or more vertically oriented walls of tray 1. The cross-sectional shape of this structure may be approximately square or rectangular, defined by two opposing walls that extend laterally outward with respect to the vertically oriented wall from which they protrude, representing the top and bottom walls of the structure. A third wall may comprise a portion of the vertically-oriented wall of tray 1 itself, arranged parallel to another vertically-oriented wall that connects the top wall to the bottom wall. Similarly to the bent top edge 42 illustrated in FIG. 12, such an end connector may comprise one or more perforations or holes 44 for receiving end inserts 33, 35 of wire supports 31.

### Wire Supports

Tray 1 also comprises one or more wire supports 31. Each wire support 31 may include at least two inserts 33 and 35 (one at each end) that flank an elongated middle portion 37, the middle portion comprising at least two bends and at least one contact segment, i.e., a length of wire that contacts an instrument to hold or support some portion of it.. The inserts 33 and 35 are in one embodiment used to anchor the wire supports 31 to one or more of the side walls 11, 13 of tray 1. A wire support 31 may form a continuous path between the side walls 11, 13 of tray 1. As shown in FIGS. 1 and 4-11, between inserts 33 and 35 the middle portion 37 of a wire support 31 may comprise various configurations of wire segments and bends to accommodate an assortment of different sterilization targets, including medical instruments and/or implants or components thereof. The level of complexity of such wire configurations may depend on the constraints of the equipment and/or processes used to form the wire supports. In particular, wire supports 31 shaped in two dimensions may primarily comprise vertically and horizontally undulating wire segments defined by approximately 90° bends and lying mainly or exclusively in a plane generally perpendicular to side walls 11, 13, whereas wire supports 31 formed in three dimensions may comprise similarly angled bends, but in addition to wire segments oriented to form vertically and horizontally undulating segments of wire traversing between side walls 11, 13, may include sire segments that are oriented in an axial direction, i.e., segments that lie in a plane perpendicular to the end walls 7, 9. Increasingly sophisticated wire forming techniques may produce more intricate wire shapes and contours defined by variously-angled bends and curves and include wire segments lying in each of the planes. Such wire support 31 configurations may include a series of high and low vertical and horizontal wire segments that together define variously-shaped contours, and also segments forming arcs, notches, bows, grooves, ridges, loops, bends, helices, or slots to receive, support and removably hold sterilization targets therein, effectively cradling, holding, and supporting all or a portion of each instrument primarily from below and from the sides. The contours may be customized to support specific instruments and/or implants. As such, the contours may comprise diverse shapes and dimensions. In some embodiments, for example, the contours may be approximately arcuate, angular, U-shaped, hemispherical, triangular, rectangular, spiraling, and/or sloping. In embodiments, the contours of wire segments may be irregularly shaped to match the profile of different instruments and/or implants at various positions along the length of each instrument. As such, the contours may comprise parallel vertical portions or non-parallel vertical portions offset from each other by varying distances. In addition to defining vertically contoured slots, wire supports 31 may extend laterally in various directions, e.g., perpendicular, parallel, and/or diagonal with respect to one or more of side or end walls of tray 1. In embodiments, the wire supports 31 may comprise horizontally-extending U-shapes, for example, the U-shapes each configured to support a sterilization target at at least one point, such that the targets hang in the U-shapes toward floor member 5 and are constrained against lateral movement. Wire supports 31 are generally configured for particular objects and sets but may also be configured to universally accommodate a generic set of instruments and/or implant components.

In the side view of tray 1 shown in FIG. 2, the objects held within tray 1 are suspended a distance above floor member 5 by four wire supports 31 traversing the tray at different locations between the end walls 7, 9. Wire supports 31 may be entirely suspended above floor member 5. Wire supports 31 may also contact floor member 5 at one or more points. In the particular configuration shown in FIG. 2, for instance, one of wire supports 31 contacts floor member 5 at segment z. Such contact points between floor member 5 and wire supports 31 may be necessary to provide additional structural support and stability when trays are moved, especially if individual wire supports 31 are configured to extend in multiple directions, resulting in wire supports 31 of greater total length. One or more contact points between wire supports 31 and floor member 5 may also be needed to support heavier objects. Wire supports 31 comprised of less rigid materials and/or thinner diameters may also require one or more contact points with floor member 5.

As shown in FIG. 2, wire supports 31, mounted along side walls 11 and 13 via mounting fixtures 39 and 41, for example, may be used to suspend instruments above the surface of floor member 5 such that assorted sterilants are able to flow beneath the instruments, thus exposing the instruments to sterilants on all surfaces. The space between floor member 5 and the instruments suspended above also facilitates sterilant flow through perforations 27 in floor member 5.

In additional designs, wire supports 31 may also pass over a top surface of various instruments and/or implants to prevent vertical movement of such objects toward cover 3, for instance. Wire supports 31 configured accordingly may be secured to tray 1 via connection means after the instruments and/or implants are initially placed on a first set of wire supports 31 that extend primarily around the bottom surfaces of the instruments. Such wire supports 31 may be utilized to further stabilize a set of instruments and/or implants upon inversion of tray 1, for example.

Wire supports 31 may be formed from wire or other thin, elongated stock of various cross-sectional shapes, sizes, and/or materials. In some embodiments, for example, wire supports 31 may comprise solid, circular cross sections. The cross-sectional shape may also be rectangular, oval, triangular, pentagonal, hexagonal, or irregular. In one embodiment, a circular or oval cross-section is used to cause wire supports 31 to contact instruments and/or implants only at points or lines of tangential contact therebetween. Such cross-sectional shapes may be solid or hollow. When bent, wire supports 31 may form smooth, rounded corners, such as those depicted in FIGS. 1-2 and 4-11, or sharper corners with more defined vertices.

The materials comprising wire supports 31 may vary. In some embodiments, for example, wire supports 31 may comprise copper, one or more metals, steel, stainless steel, or any combination thereof. In a particular embodiment, 300 series stainless steel may be used. In additional or alternative embodiments, wire supports 31 may be galvanized steel or coated with various materials, e.g., sealants, to further protect wire supports 31 from damage and/or to minimize frictional forces between wire supports 31 and the instruments and/or implants placed thereon.

The cross-sectional diameter of each wire support 31 may also vary. In some embodiments, the diameter or width (if the cross-section is not generally round), may be increased to support heavier objects, or decreased for lighter objects. The diameter of the wire may also be decreased to accommodate a greater number of objects within tray 1 and/or to minimize the size of the surface contact areas between the instruments and wire supports 31. In some embodiments, the cross-sectional thickness of wire supports 31 may range from about 0.05 to about 0.3 inches, about 0.075 to about 0.25 inches, about 0.1 to about 0.2 inches, about 0.125 to about 0.175 inches, or about 0.14 to about 0.16 inches.

Due in part to their narrow cross-sectional diameter, wire supports 31 do little or nothing to disturb, impede, or otherwise direct the flow of air, steam, and/or various other sterilants circulating throughout tray 1 during sterilization processes, in particular as compared to more planar support elements of conventional trays. As a result, instruments and/or implants sterilized within tray 1 may be thoroughly sterilized on a consistent basis. The narrow diameter of wire supports 31 also minimizes the internal bulk of tray 1, which may decrease the overall weight of the tray and enable tray 1 to accommodate a greater number of objects at one time. Such small, compact, and dense wire supports 31 may also be less vulnerable to cracking during intense heat, pressure, and chemical treatments. With a smaller surface area compared to other support structures in the prior art, the likelihood of wire supports 31 becoming contaminated after sterilization may also be reduced.

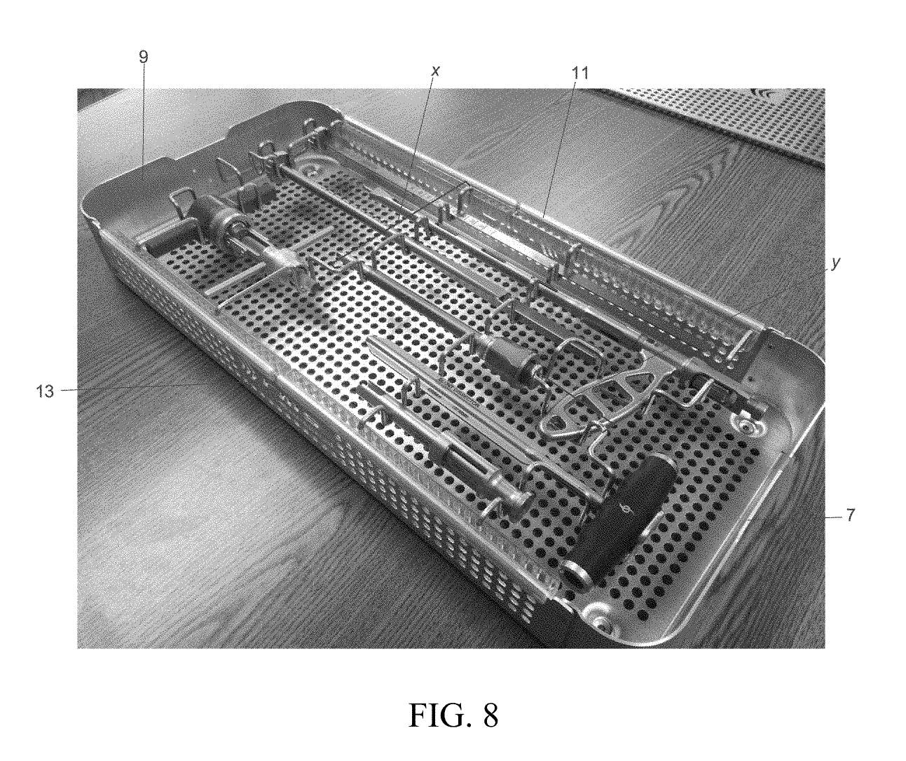

FIG. 8 is another view of tray 1 holding various instruments via multiple wire supports 31. In particular, to support both ends, or both ends and a middle portion of an elongated instrument, a separate wire support may need to be configured at each of the ends and a middle. As shown, wire supports 31 may be configured to restrict both lateral and longitudinal movement of the objects held within tray 1 by providing differently-shaped contours along different points of each object. In particular, wire supports 31 may be customized to encompass particular features of various objects, e.g., handles, knobs, projections, protrusions, etc., having contact surfaces facing different directions. Some embodiments, for example, feature segments of wire extending in a parallel direction with respect to end walls 7 and 9, such as segment x in FIG. 8. The vertically undulating contours of such segments restrict movement of the suspended objects in a lateral direction toward each of side walls 11 and 13. Embodiments may also feature wire segments oriented at various additional angles with respect to end walls 7 and 9, such segments being specifically positioned to restrict longitudinal movement of the suspended objects toward end wall 7 or 9 by barricading instrument features not oriented parallel to side walls 11 and 13. An exemplary wire segment arranged to prevent longitudinal movement of an object is represented by segment y in FIG. 8. As illustrated, segment y defines a contour with vertical portions that prevent the instrument held therebetween from sliding toward or away from end wall 9. Using one or more wire supports 31 comprised of a combination of parallel, perpendicular, and/or diagonal segments, diversely-shaped medical instruments and/or implants may be suspended securely within internal cavity 29 without making contact with other instruments concurrently held therein.

As illustrated in FIG. 8, the structure of wire supports 31 may be customized in multiple ways within tray 1. In particular, the individual contours within each wire support 31 may be customized to match specific sterilization targets. The collective group of contours comprising one middle portion 37 of each wire support 31 may also be customized. For example, two wire supports 31 may comprise the same set of individual contours, but arranged in a different order. In addition, the overall organization and collection of wire supports 31 within a particular tray 1, each having one middle portion 37 and two inserts 33 and 35, may be further customized to correspond to a particular group of sterilization targets. Thus, many organizational permutations are possible by customizing the shape and/or arrangement of wire supports 31, imparting the trays 1 disclosed herein with the high degree of design flexibility necessary to accommodate a wide range of sterilization targets.

As shown in FIGS. 5 and 6, wire supports 31 may be secured to mounting fixtures 39 and 41, or other connection means, at various points along the length of tray 1. In some embodiments, inserts 33 and 35 of a single wire support may be secured directly across from each other by receiving slots or holes 43 positioned at the same longitudinal position with respect to end walls 7 and 9. In other embodiments, inserts 33 and 35 may not align longitudinally, secured instead to receiving slots or holes 43 at different distances from end walls 7 and 9.

In the particular embodiments illustrated in FIGS. 5 and 6, each wire support 31 comprises two inserts 33 and 35 (one at each end) flanking one middle portion 37, with each insert configured for insertion into one of receiving slots or holes 43. In other embodiments, each wire support 31 may comprise more or less inserts configured for insertion into one of receiving slots or holes 43. For example, a wire support 31 may be comprised of only one insert removably secured via connection means to tray 1. In such embodiments, a second insert may be free standing, unattached to various connection means. In other embodiments, both inserts 33 and 35 may be permanently fixed to any combination of floor member 5, cover 3, side walls 11 and 13, and/or end walls 7 and 9, leaving no inserts configured for slidable insertion into receiving slots or holes 43. In still other embodiments, one wire support 31 may comprise three or more inserts. According to such embodiments, the middle portion 37 of wire support 31 may comprise a branched configuration with multiple inserts at multiple ends.

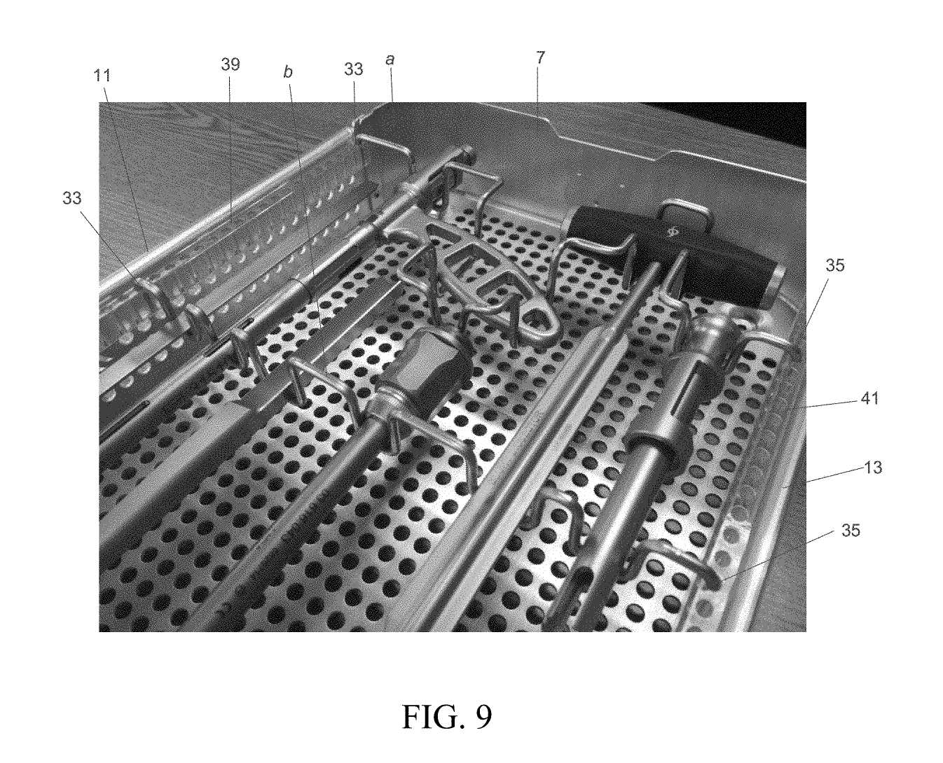

FIGS. 9-11 illustrate longitudinally successive views of a particular collection of wire supports 31 used to suspend a specific group of instruments within tray 1, highlighting the varied structure of each wire support along the length of each instrument. FIG. 9 shows a first end of tray 1 and two wire supports, a and b. Wire support a serves primarily to secure the terminal portions of each instrument shown. In this particular example, such terminal portions comprise variously-configured handles, knobs, and/or protrusions around which wire support a is configured. Insert 33 of wire support a is inserted within the receiving slot or hole 43 of mounting fixture 39 closest in proximity to end wall 7, while corresponding insert 35 is inserted within a receiving slot or hole 43 further from end wall 7 within mounting fixture 41. As shown, wire support a comprises an assortment of variously-configured contours and bends customized to secure the instruments depicted in this example.

In contrast to wire support a, wire support b is entirely parallel to end wall 7, featuring inserts 33 and 35 secured to receiving slots or holes 43 equidistant from end wall 7. The contours defined by wire support b primarily restrict lateral movement of the instruments held therein with respect to side walls 11 and 13.

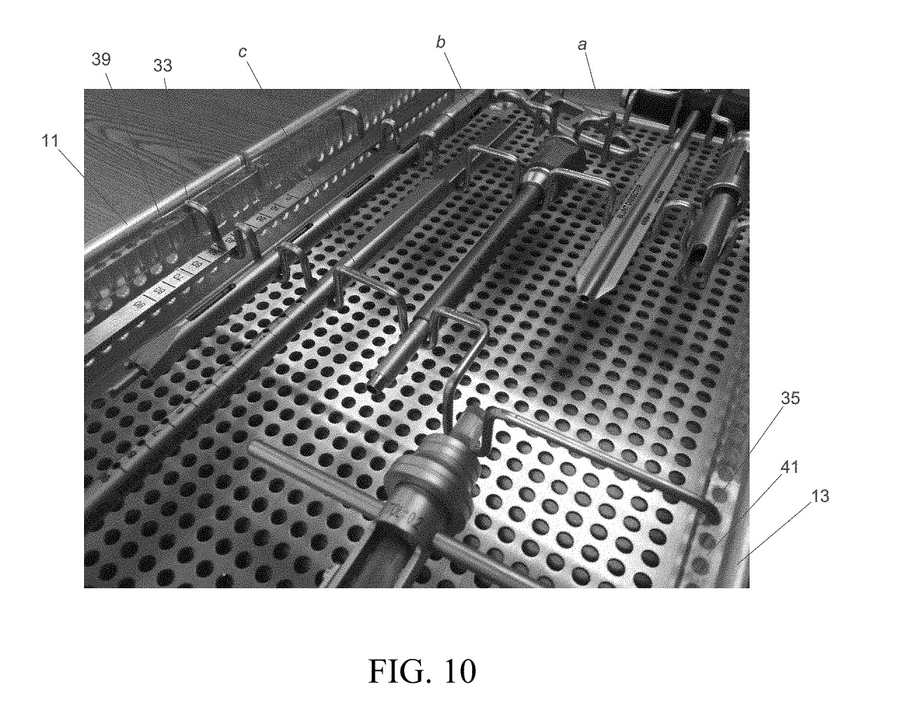

FIG. 10 shows wire support c secured to a middle portion of tray 1. Like wire support a, wire support c comprises inserts 33 and 35 inserted within receiving slots or holes 43 at different longitudinal positions. As illustrated, wire support c supports the more distal portions of several of the instruments depicted in FIG. 9, as well as the terminal portion of another instrument positioned opposite the first end of tray 1.

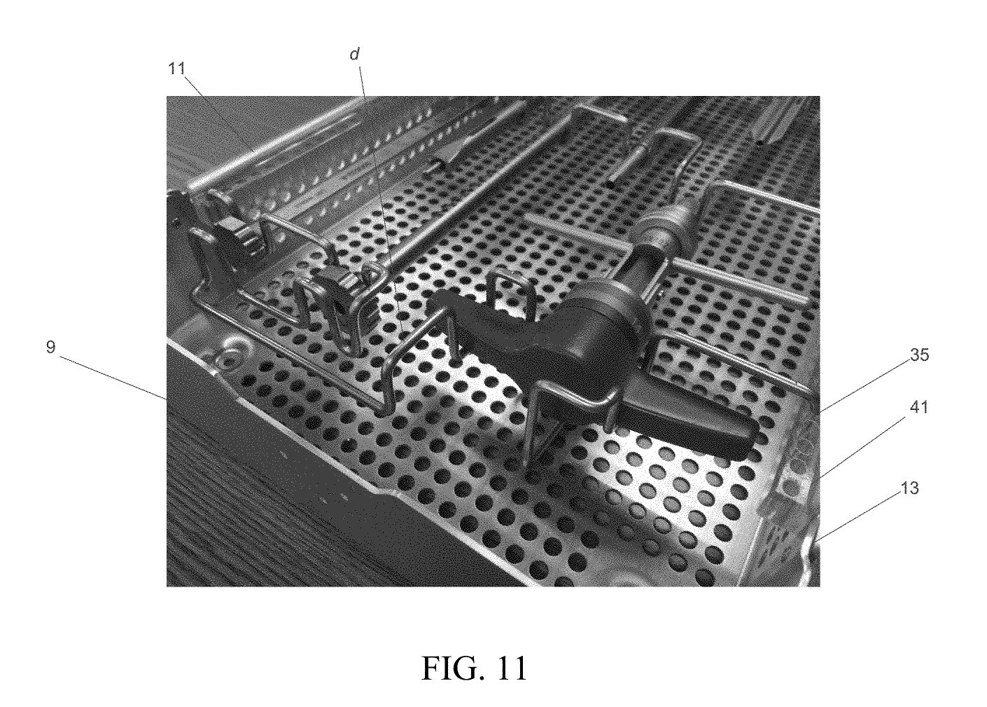

FIG. 11 depicts a second end of tray 1 near end wall 9, including wire support d. Wire support d comprises a distinct configuration tailored to the specific shape of the knob and handle features respectively capping the terminal portions of the three instruments shown. Each instrument is prevented from sliding into end walls land 9 and side walls 11 and 13 by vertically oriented segments of wire support d, each vertically oriented segment connected by horizontally extending segments oriented parallel or perpendicular to side wall 9 Thus, wire support d restricts lateral and longitudinal movement of the instruments via a series of wire bends, the bends collectively defining wire segments extending in various vertical and horizontal directions. In greater particularity, wire support d extends horizontally away from side wall 11 for a short distance until a first bend in the wire extends downward at an approximately 90° angle to accommodate a knob portion of the first instrument proximate to side wall 11. In a series of approximately 90° bends, wire support d extends beneath the knob portion and back upward, away from floor member 5.

The vertical segments defined by the series of bends restrict the first instrument from moving toward side walls 11 and 13, and further restrict the knob portion from moving toward end wall 7 (not shown). After supporting the knob portion of a second instrument in similar fashion, wire support d extends back toward side wall 11, comprising a series of bends collectively defining vertically-undulating contours that prevent the knob portions of the first two instruments from moving toward end wall 9. Wire support d then extends horizontally toward side wall 13 for a distance, bending vertically upward and then horizontally away from side wall 9 to accommodate the handle portion of a third instrument. A series of additional bends define wire segments extending vertically and horizontally to abut the handle portion from different sides, thereby restricting movement of the handle portion toward each side and end wall. The final segment of wire support d extends horizontally toward side wall 13, where end insert 35 couples with mounting fixture 41. The customized collection of bends, loops, and contours comprising wire support d may embody a representative example of the specialized fit provided by the wire supports 31 disclosed herein. As shown, various segments of each wire support 31 may be oriented in various directions to accommodate different instrument and/or implant features. Such segments may comprise contours, bends, slots, or loops of various shapes, widths, and/or depths to suspend the instruments and/or implants a distance above floor member 5 within cavity 29. Despite the custom fit of the wire supports 31 with respect to the sterilization targets held thereon, and the resulting restriction in movement of such instruments, the contact surfaces between the wire supports 31 and the instruments and/or implants may be minimized.

As shown in each of FIGS. 9-11, the contact points between wire supports 31 and the instruments held thereon may be reduced in comparison to other designs in the prior art. In particular, the narrow thickness of wire supports 31, their circular cross-section, and their customized shape with respect to the objects they are used to secure minimizes the surface area of the contact points between the two components. By decreasing the size and/or number of contact points, more of the surface area of the objects held within tray 1 remains exposed to sterilants and/or fewer surfaces areas are blocked from exposure by wire contact, thus improving the effectiveness of sterilization processes.

### Methods of Forming

Floor member 5, cover 3, side walls 11 and 13, and end walls 7 and 9 of tray 1 may be manufactured from a single sheet of metal, for example, by stamping and bending methods. In other embodiments, the various components of tray 1 may be manufactured from multiple pieces or sheets of metal. Such components may be coupled together via rivets, screws, pins, or other mechanical fasteners via heat, sonic, laser, or spot welding. Alternatively, tray 1 can be manufactured in substantially unitary form by bending or other forming operations on a sheet of material with the desired perforations. Suitable materials for tray 1 and cover 3 include, but are not limited to, one or more metals, steel, stainless steel, plastics and other durable polymers, composite materials, and combinations thereof. Preferably, the materials have or can be provided with the desired perforations and are able to withstand the elevated heat and pressure conditions, as well as chemical treatments, commonly associated with sterilization processes.

Connection means comprising mounting fixtures 39 and 41 may be comprised of various materials. Suitable materials include, but are not limited to, one or more metals, steel, stainless steel, plastics and other durable polymers, composite materials, and combinations thereof. In one particular embodiment, mounting fixtures 39, 41 may be formed from Radel® polyphenylsulfone. In additional or alternative embodiments, various sulfone polymers may be utilized. The material may be the same or different as the material comprising the other structural components of tray 1. In one embodiment, mounting fixtures 39, 41 may be formed by injection molding one or more polymer compositions. Various adhesives, hooks, and/or fasteners, e.g., screws or bolts, may be utilized to secure the connection means to tray 1, as necessary.

Wire supports 31 may be manufactured by various methods of bending wire to assume particular conformations, which may require the application of heat and/or use of wire-bending machinery. Such machinery may receive stocks of wire fed directly into the machinery. Such machinery may then introduce one or more bends into the wire in two or more dimensions and at specific locations along the wire length. The machinery may further cut the wire at predefined lengths.

As seen in FIG. 12 a wire support 31 may be constructed from a small set of elements that nonetheless allows a wide variety of designs. To provide holding and support for an instrument 1210 with a cylindrical cross-section (shown in phantom in FIG. 12), the support may include one horizontal wire contact segment 1220 and two associated vertical wire contact segments 1222 and 1224, each of which is connected to horizontal contact segment 1220 by a bend 1223 or 1225, respectively. Each contact segment is designed to be in direct contact with or a small clearance (e.g., 1/16 to 1/18 inches) away from direct contact with a contact point on a surface of the instrument 1220, e.g., contact point 1227 of contact segment 1222. Wire connection segments are also part of wire support 31. For example, connection segment 1240 is joined by a bend to vertical contact segment 1222 and connection segment 1242 is joined by a bend to vertical contact segment 1224. Connection segment 1240 is also joined by a bend to a wire segment that forms insert 33.

As can be seen, a contact segment or a connection segment can be fully defined by its length and its end points (represented as dots in FIG. 12) in the sterilization space or cavity 1250 defined by the tray in which support 31 is to be used. More specifically, definition of a contact segment or a connection segment can be based on the diameter of the wire and the end points of a center line of the wire. A bend can be similarly defined, with the addition of the radius of the arc between its end points. In the example of FIG. 12, only bends of ninety degrees are present, but it will be seen that a bend of 45, 50, 60 or 110, 120, 130 degrees or many other acute or obtuse angles can be formed. Limits on such angles may arise with some wire and certain bending machines.

The data needed to design a support 31 (which will usually be one of a set of supports) with contact segments, connection segments, and bends is a dimensional design description of the sterilization target(s) to be held and supported and a dimensional design description of the tray, which in turn defines the sterilization space or cavity 1250 of the tray in which an instrument or other sterilization targets is to be held and supported by one or more supports 31. With these data, a designer can select for a given instrument its location in the sterilization space or cavity 1250 of the tray. Once the location is known, the various contact segments and contact points for holding and support of the instrument can be selected and defined. For example, the horizontal contact segment 1220 may be selected to hold and support the instrument above the floor of the tray, while the two vertical contact segments 1222, 1224 may be selected to provide holding and support against lateral movement.

While a skilled designer in possession of the above dimensional design description data can design a wire support starting from a first insert (e.g., insert 33 in FIG. 12) and leading through bends and connection segments to a first contact segment and through further bends, connection segments and contact segments to a second insert (not shown in FIG. 12), the particular conformations of wire supports 31 also may be designed with the aid of a computer. In particular computer aided design (CAD) programs (e.g., the software known as SolidWorks from SolidWorks Corporation) may be provided with a dimensional design description of the sterilization targets comprising one or a set of instruments that it is desirable to sterilize and later use together (often in a prescribed sequence) and a dimensional design description of the tray for this set, which in turn defines the sterilization space or cavity 1250 of the tray in which the instrument set is to be held and supported. The CAD software may be utilized to design and provide shape and dimension specifications for a particular set of wire supports 31 that match a given set of instruments and/or implants while using efficiently the length of wire. In some examples, two-dimensional or three-dimensional CAD software may be used to electronically model and manipulate a set of graphically-displayed, virtual sterilization targets within a virtual sterilization space. In particular, the software may “suspend” a specified set of virtual sterilization targets above floor member 5 within internal cavity 29 (see FIG. 1), the cavity initially devoid of any wire supports. Using the CAD software and dimensional design descriptions of the tray and the sterilization targets, the targets may be arranged at specific locations within the virtual cavity according to their order of use in a corresponding medical operation or task, such that the targets may be progressively removed from the tray in the order with which they are arranged, thus facilitating easy access to the tray and removal of the sterilization targets. The sterilization targets may also be positioned so as to allow efficient flow of sterilization fluids around all surfaces to be sterilized and/or to reduce risk of damage to more delicate instruments or their features.

After defining the specific location and arrangement for sterilization targets within the sterilization space corresponding to an actual tray, which leads to an x, y, z dimensional map of each target in the virtual cavity, a user may define the specific desired contact points for holding and supporting the sterilization targets, by location in the virtual cavity of desired contact points between a target and a wire contact segment that contacts a selected point of the target. (In some cases, depending on the shape of the surface of a sterilization target, the “contact point” may be a contact line or a contact surface; however, to facilitate contact of sterilization fluid with instrument surfaces, a limited contact line or a contact surface is preferred. In the following description, “contact point” may also mean a contact line or a contact surface.). This then allows a support wire designer to choose the contact segments needed in a particular wire support 31 and the bends needed to connect segments, starting with a wire support insert at a particular location on a connection means, to position a contact segment that will contact the selected point of the target.

The path of the wire support to and from a wire segment that will contact the selected contact point of the target, may be constrained by the minimum bending radius available for a particular wire diameter and material used and the specific bending capabilities of the wire bending machine that is to be programmed. However, with available bends between 80 and 179 degrees essentially all desired configurations can be built (keeping in mind that adjacent wire supports can contribute to the holding and support of a common instrument for which both wires provide contact segments. The design process may entail identifying multiple contact points based on the specific structural configuration of each target. The multiple contact points on an elongated target may be assigned to separate wire supports. Bends and the points where they begin and end may be specified, by the direction radius and radius center-point of each bend, such that the various contours defined by such bends lead into the contact segments providing the selected contact points of a given wire support with each target the support is designed to contact. The number of wire supports necessary to hold and support each target at its selected location may also be specified. The design of a target of known dimensions and located at particular x, y, z position selected in the sterilization space defined for a specific tray may be translated into a design of one or more wire supports configured to hold and support the target, with a beginning insert at one connection means and an ending insert at another connection means.

For most applications, the wire supports needed can each be defined in terms of: (1) the known configuration for each end (e.g., a short vertical segment for receipt in a receiving slot or hole 43 of mounting fixture 39, 41, or other end connector structure); (2) the shape or path of the wire support as it extends between the sides of a tray, defined in terms of 90 degree angle bends (or other angles) and the orientation of such bends (radius and radius center-point); and (3) the length of the contact and connection segments between bends. This can result in a relatively straightforward design file for the control of a wire bending machine that will produce a complex shape. The design file may be provided in a format that is an acceptable input format for a specific wire bending machine.

For wire supports configured in one plane, a two-dimensional wire bending machine able to handle the desired wire stock is sufficient, such as a Model 4S-6 from Mang Systems, Inc. of Mathews, N.C. or a Model AFE-2Dx from Automated Industrial Machinery, Inc. of Addison, Ill. For a wire support that is not in one plane, a three-dimensional wire bending machine able to handle the desired wire stock is needed, such as a Model 6S-6 from Mang Systems, Inc. of Mathews, N.C. or a Model AFM 3DX from Automated Industrial Machinery, Inc. of Addison, Ill. For more complex shapes, a wire bending machine that can bend angles other than 90 degree angle bends and/or form arcs, curves or other more complex shapes may be needed. Such a machine may have additional axes, e.g., up to 16 axes, for bending the wire into more complex shapes.

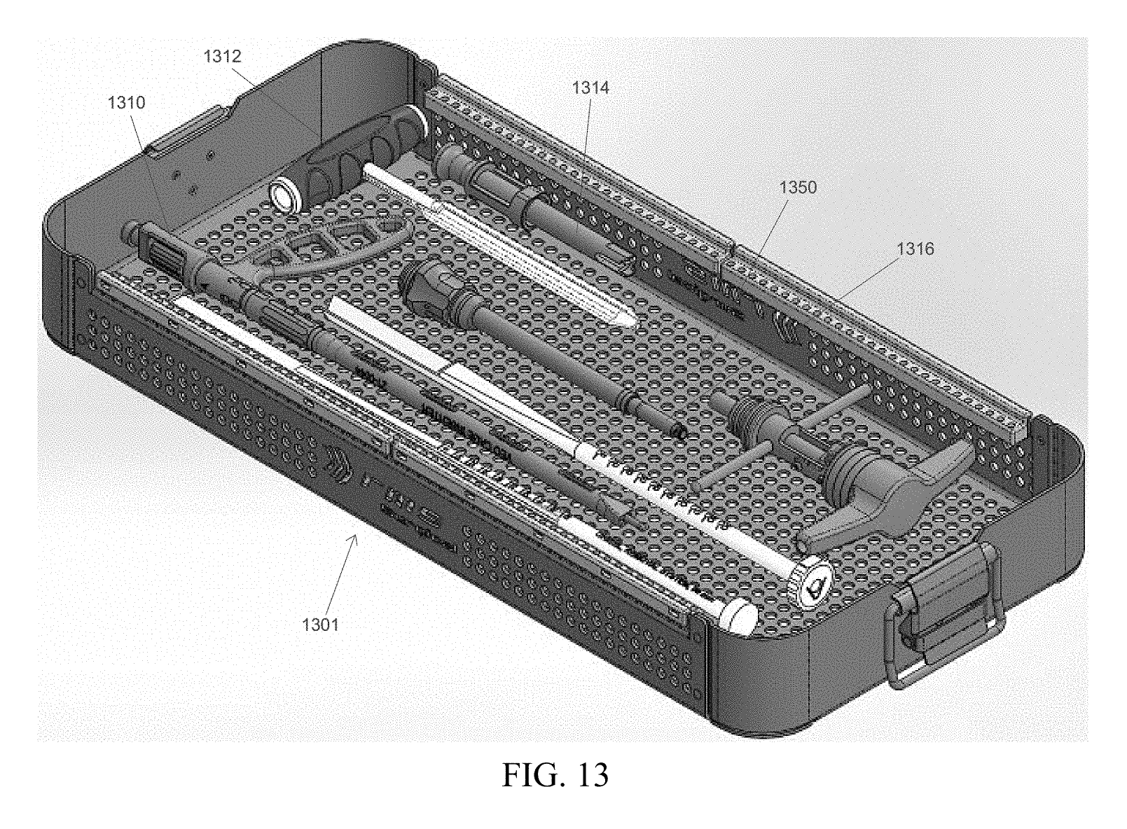

An example as shown in FIGS. 13-15 assists in understanding the design process for a wire support, which includes the following steps: using a computer aided design tool programmed with a dimensioned design description for a tray for holding sterilization targets, including the sterilization space, defining a location in the sterilization space for at least one sterilization target; using a computer aided design tool programmed with a dimensioned design description for the at least one sterilization target, identifying at least one contact point on the sterilization target, for supporting and/or holding the sterilization target at its location in the sterilization space; using a computer aided design tool programmed with the bending capabilities of wire bending machine and a wire specification, defining a wire path between two connection means associated with at least one of a plurality of vertically oriented tray walls for securing each end of the at least one wire support to the tray, said wire path defining a wire configuration extending between two end points of at least one wire contact segment and passing through the at least one contact point and at least one wire connection segment at each end of the wire contact segment, defining at least one connecting bend in the wire path; storing in a storage medium a wire path design file including the location of a plurality of end points of the contact segments and connection segments and the radius or other specification of the connecting bends, the wire path forming a continuous path between the two connection means; transmitting to a control unit of the wire bending machine the wire path design file; and causing the wire bending machine to perform bending of a wire supplied to the bending machine as defined by the wire path design file, including forming an insert for each of the connection means by bending and cutting wire at each end of the wire path.

FIG. 13 shows a magnified isometric top view of the tray of FIG. 7 (here referenced as 1301), with all wire supports visible in FIG. 7 removed. Thus, FIG. 13 shows the locations of a set of medical instruments (one type of sterilization target) in a sterilization space 1350 defined by the tray. In the example of FIG. 13, the instruments of interest for the design of one wire support are referenced as 1310, 1312, 1314 and 1316. The dimensions of the sterilization space and all the instruments are provided as data for the CAD software tool. The designer will use CAD software to position each sterilization target in the available sterilization space 1350.

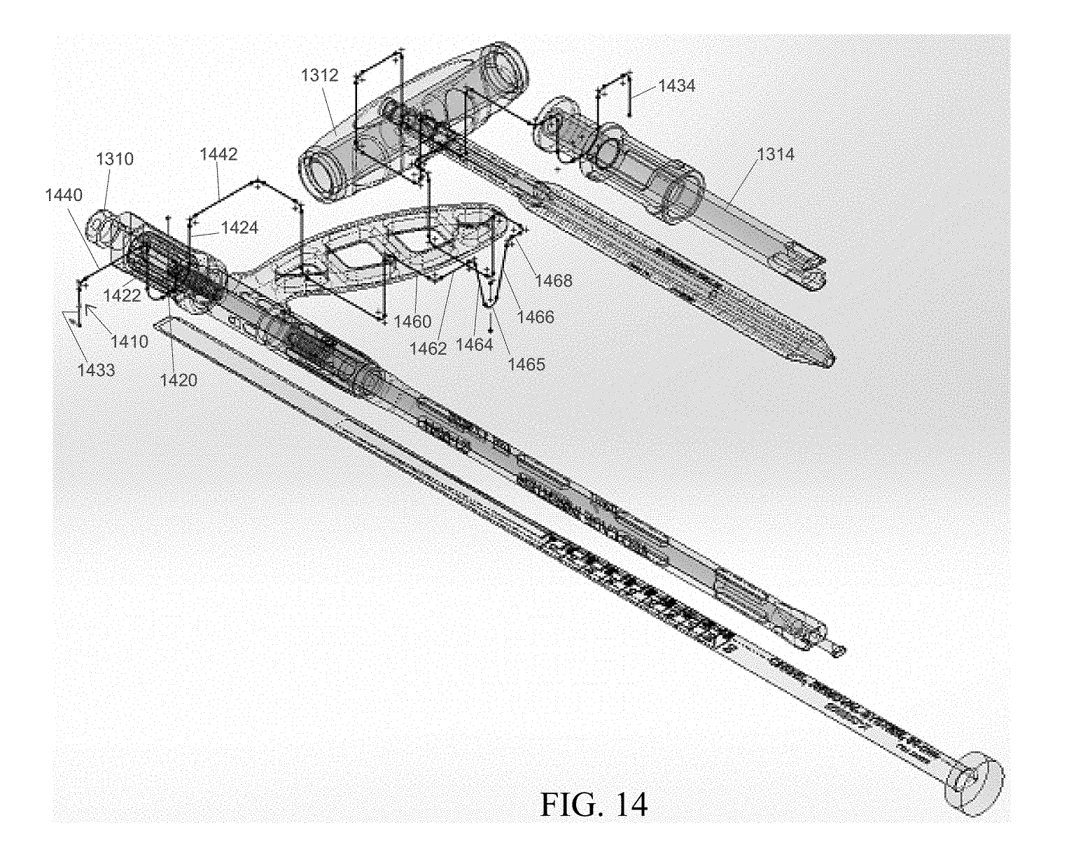

FIG. 14 is magnified isometric top view of the plurality of medical instruments of FIG. 13 as located in the sterilization space defined by tray 1 (which for clarity is not shown) and showing a wire path 1410 for the leftmost wire support in FIG. 7, as defined by the location of end points of support segments, connection segments and bends in the sterilization space defined by the tray shown in FIG. 13. As can be seen, only a center-line of the wire path is shown; for design purposes the wire is initially assumed to have no thickness. The wire path 1410 begins with an insert segment 1433 and after a 90 degree bend in a vertical plane leading to a horizontal connection segment 1440, in turn leading to a further 90 degree bend in a vertical plane, leading to a descending vertical contact segment 1422. A further 90 degree bend in a vertical plane leads to a horizontal contact segment 1420 under a portion of instrument 1310, providing support at a contact point. A further 90 degree bend in a vertical plane leads to an ascending vertical contact segment 1424, generally parallel to vertical contact segment 1422. Thus, the vertical contact segments 1422, 1424 provide lateral holding on either side at opposing contact points on a portion of instrument 1310, points lying in the same plane, transverse and extending between tray side walls. The wire path continues with further connection segments, contact segments and bends as seen in FIG. 14.

The portion of the wire path that includes connection segment 1460 has an orientation that is in the longitudinal direction of the tray, and leads via a bend to a connection segment 1462 that has an orientation that is in the transverse direction of the tray. Connection segment 1462 leads via a bend to an angled descending contact segment 1464 and an arcuate bend positioned at a defined height above the tray floor. The arcuate bend leads into an angled ascending contact segment 1466 and an arcuate bend leading to contact segment 1468. These segments are part of this wire path, but are intended to provide holding of the enlarged end of instrument 1316 of FIG. 13 (in center of tray), which is not shown in FIG. 14. Once the wire path centerline for support 1410 is complete from end to end, the wire thickness can be added to the design. In effect the center-line is “extruded” to be a desired thickness, with the CAD device making the appropriate adjustments to add volume to the geometry of the centerline.

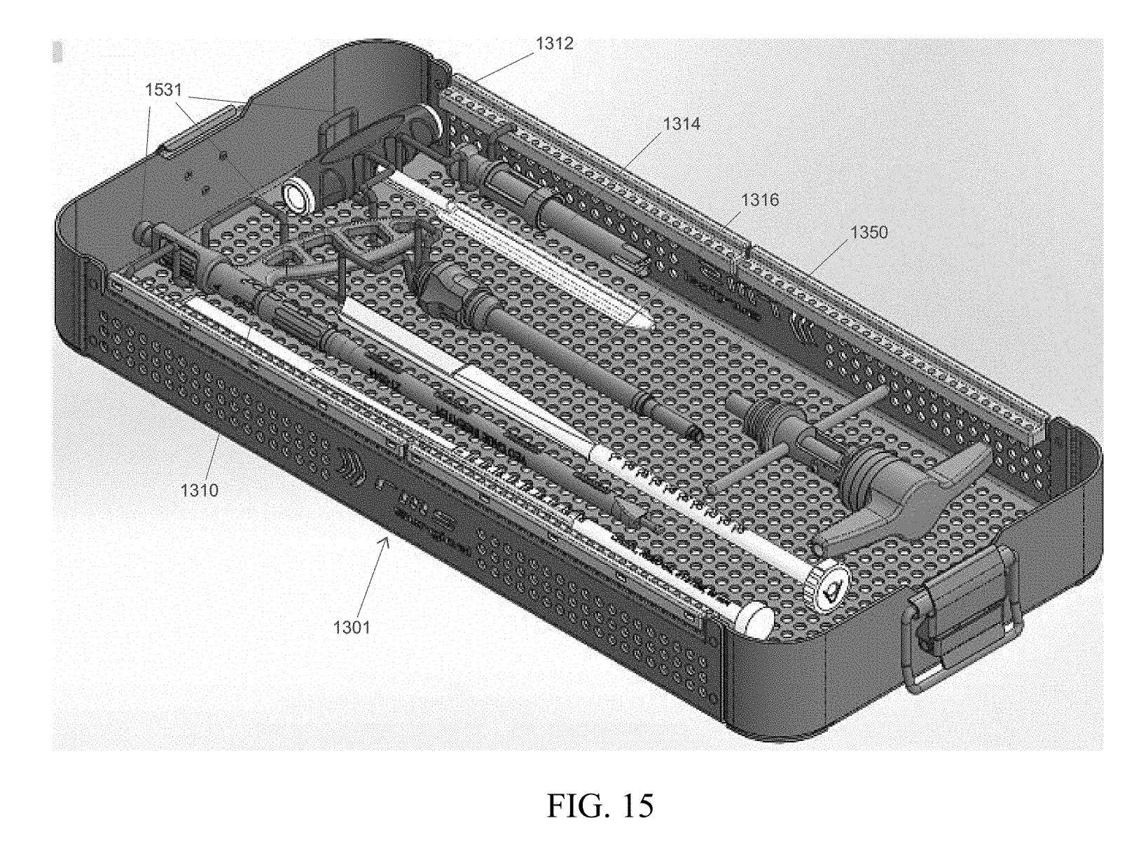

FIG. 15 is magnified isometric top view of the plurality of medical instruments of FIG. 14 (with additions from FIG. 13) as located in the sterilization cavity defined by tray 1301 and showing the complete leftmost wire support 1531 also shown in FIG. 7, as formed by a wire bending machine programmed with the wire path of FIG. 14. FIG. 15 shows how the complete wire support 1531 extends across tray 1301 and provides holding and support for instruments 1310, 1312 and 1314. FIG. 15 also shows how the angled descending and ascending contact segments 1464 and 1466 and the bend that joins them provide holding of the enlarged end of instrument 1316 of FIG. 13 and FIG. 15.

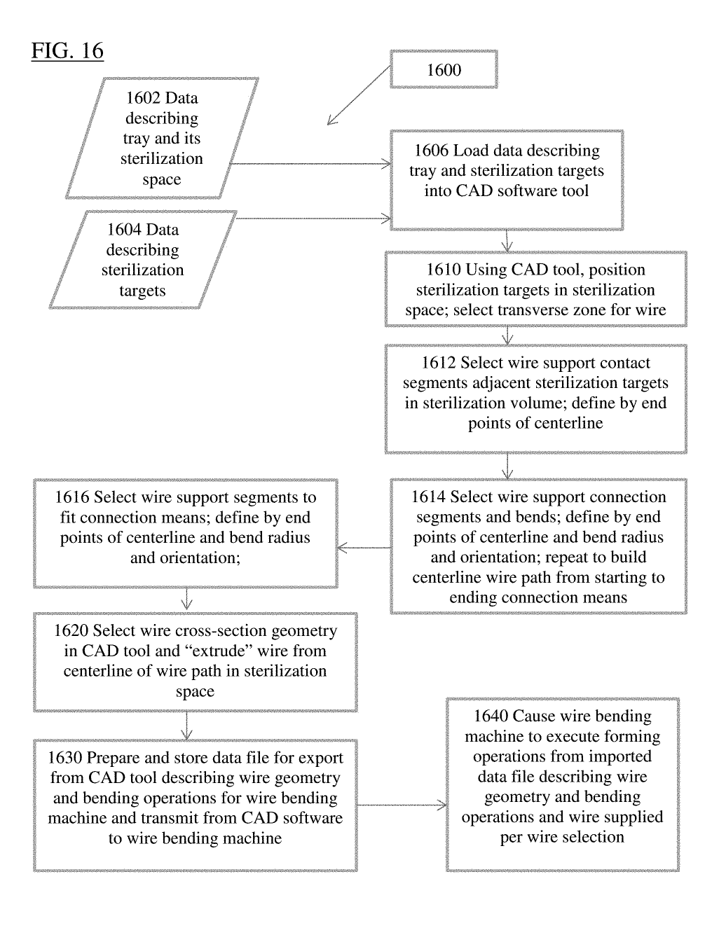

FIG. 16 shows in schematic diagram form a flow chart 1600 for one embodiment of a process for preparing a design file for a support to be made from wire and used in a tray as described above and executing that design in a wire bending machine. The steps 1602 through 1640 of the process as shown are generally as described above. The designer will use this process for each wire support, after selecting a transverse zone of the tray where the wire support will extend between the side walls of the tray. It will be seen however that a wire support may be designed to start and end at the same side wall if required to a certain target configuration. Such a support may be cantilevered from the connection means on the side wall or include segments that for a support extending to the tray floor.

Ann appropriately formatted file with a description of a specific set of instruments and/or implants or components thereof may be stored and used to prepare an appropriately formatted file with a description of a specific set of wire supports for these instruments and/or implants or components. This file may be input into a process control component of the wire-forming machinery. Such machinery may then bend a stock wire to provide one or more wire supports 31 configured for attachment within tray 1 and custom fit to the particular set of instruments and/or implant components input by a user.

As used herein, the term “targets” refers primarily to assorted medical instruments and/or implants or components thereof; however, “objects” may also include various devices, equipment, and/or waste materials that may necessitate one-time or periodic sterilization.

While the tray and method have been described with reference to exemplary embodiments, it will be understood by those skilled in the art that various changes can be made and equivalents may be substituted, without departing from the spirit and scope of the disclosure. In addition, modifications may be made to adapt the teachings of the invention to particular situations and to use other materials, without departing from the essential scope thereof. The tray and method are thus not limited to the particular examples that are disclosed here, but encompass all of the embodiments falling within the scope of the claims.

What is claimed is:

1. A tray for holding sterilization targets comprising:
a perforated floor member;
a plurality of vertically oriented walls connected to the floor member around a perimeter of the floor member such as to define an internal cavity, wherein one or more of the walls may be perforated;
at least one wire support for removably holding and supporting against lateral or longitudinal movement at least one sterilization target within the internal cavity, wherein each wire support comprises two ends and a middle portion, the middle portion comprising at least two bends in a wire from which the support is made; and
connection means associated with at least one of the plurality of vertically oriented walls for securing each end of the at least one wire support to the tray.

2. The tray of claim 1, further comprising a cover piece removably attached to the vertically oriented walls and positioned opposite the floor member.

3. The tray of claim 2, wherein the cover piece is configured to snap onto the plurality of vertically oriented walls.

4. The tray of claim 1, further comprising at least one caddy configured to attach to the tray via the connection means.

5. The tray of claim 4, wherein the caddy comprises one or more slots, holes, baskets, and/or cavities configured to removably hold one or more small sterilization targets therein.

6. The tray of claim 4, wherein the caddy comprises one or more perforations.

7. The tray of claim 1, wherein the at least one wire support is configured to suspend at least one sterilization target a distance above the floor member such that sterilants employed during sterilization processes contact each surface of the sterilization target and a sterilant flow within the tray is improved relative to trays lacking a distance between the floor member and at least one sterilization target suspended thereabove.

8. The tray of claim 1, wherein the at least one wire support comprises various contoured, bent, or curved portions customized to removably hold or support a specific set of sterilization targets such that the sterilization targets remain stationary within the internal cavity.

9. The tray of claim 1, wherein the at least one wire support does not contact the perforated floor member.

10. The tray of claim 1, wherein the tray is configured to simultaneously hold about 1 to about 10 sterilization targets.

11. The tray of claim 1, wherein the tray comprises at least two or more wire supports configured to support a sterilization target at two or more contact points along its length.

12. The tray of claim 1, wherein the tray comprises at least one wire support supported on at least two of the plurality of vertically oriented walls.

13. The tray of claim 1, wherein the at least one wire support is comprised of one or more materials selected from the group consisting of one or more metals, steel, stainless steel, galvanized steel, copper, aluminum, one or more polymers, and any other materials suitable for sterilization.

14. The tray of claim 1, wherein the at least one wire support has a cross-sectional thickness of about 0.05 to about 0.3 inches.

15. The tray of claim 1, wherein the tray comprises four vertically oriented walls, including two oppositely-facing side walls and two oppositely-facing end walls.

16. The tray of claim 16, wherein the tray comprises a plurality of perforations on each of the two side walls, the floor member, and a cover piece.

17. The tray of claim 1, wherein the connection means comprise at least one mounting fixture further comprising a plurality of receiving slots or holes, the mounting fixture arranged along one or more walls, wherein the at least one wire support is removably secured to the mounting fixture by inserting an end of the wire support into one of the plurality of receiving slots or holes.

18. The tray of claim 17, wherein the tray comprises two mounting fixtures, each mounting fixture secured along a vertically-oriented wall.

19. The tray of claim 17, wherein each of the receiving slots is compatible with ends of variously configured wire supports, such that the wire support ends may be inserted into different receiving slots or holes as desired by a user.

20. The tray of claim 17, wherein the receiving slots or holes each comprise a depth of about 0.1 to about 2 inches.

21. The tray of claim 17, wherein the receiving slots or holes are cylindrical.

22. The tray of claim 1, wherein the tray is configured to the internal cavity for steam and/or chemical sterilization processes.

23. A tray for holding sterilization targets comprising:
a perforated floor member;
a plurality of vertically oriented walls connected to the floor member around a perimeter of the floor member such as to define an internal cavity, wherein one or more of the walls may be perforated;
at least one wire support for removably holding and supporting against lateral or longitudinal movement at least one sterilization target within the internal cavity, wherein each wire support comprises two ends and a middle portion, the middle portion comprising at least two bends; and
at least one end connector configured to secure an end of at least one wire support to the tray.

24. The tray of claim 23, wherein the at least one end connector is comprised of one or more structures selected from the group consisting of a perforated, bent top edge of a vertically oriented wall, a receiving space for a latch or pin, and other connecter structure suitable for securing the at least one wire support to the tray.

25. A method of forming a wire support for use in a tray for holding sterilization targets comprising: (i) a perforated floor member; and (ii) a plurality of vertically oriented walls connected to the floor member around a perimeter of the floor member, wherein one or more of the walls may be perforated and the floor member and walls together define a sterilization space enclosed by the tray, comprising:
using a computer aided design tool programmed with a dimensioned design description for the tray for holding sterilization targets, including the sterilization space, defining a location in the sterilization space for at least one sterilization target;
using a computer aided design tool programmed with a dimensioned design description for the at least one sterilization target, identifying at least one contact point on the sterilization target, for supporting or positioning the sterilization target at its location in the sterilization space;
using a computer aided design tool programmed with the bending capabilities of a wire bending machine and a wire specification, defining a wire path between two connection means associated with at least one of the plurality of vertically oriented walls for securing each end of the at least one wire support to the tray, said wire path defining a wire configuration extending between two end points of at least one wire support segment and passing through the at least one contact point and at least one wire connection segment at each end of the wire support segment defined by a connecting bend in the wire path;
storing in a storage medium a wire path design file including the location of a plurality of end points of the support segments and connection segments and a radius or other specification of the connecting bends, said wire path forming a continuous path between the two connection means;
transmitting to the control unit of the wire bending machine the wire path design file; and
causing the wire bending machine to perform bending of a wire supplied to the bending machine as defined by the wire path design file, including forming an insert for each of the connection means by bending and cutting wire at each end of the wire path.

26. The method of claim 25, wherein defining a location in the sterilization space for at least one sterilization target comprises arranging two or more sterilization targets within the sterilization space according to an order of use in a corresponding medical operation or task, such that the targets may be progressively removed from the tray in the order with which they are arranged.

27. The method of claim 25, wherein defining a location in the sterilization space for at least one sterilization target comprises suspending the at least one sterilization target a distance above the perforated floor member within the sterilization space.

28. The method of claim 25, wherein the at least one contact point comprises a contact line or a contact surface.

29. The method of claim 25, wherein defining a wire path further comprises specifying a wire thickness.

30. The method of claim 25, wherein the computer aided design tool comprises two-dimensional or three-dimensional modeling capabilities.

31. The method of claim 25, wherein the radius or other specification of the connecting bends comprises points where the connecting bends begin and end, a direction radius and a radius center-point of each bend, such that various contours defined by the connecting bends lead into contact segments and connection segments.

32. The method of claim 25, further comprising selecting a number of wire supports necessary to hold and support the at least one sterilization target at its defined location.

33. The method of claim 25, wherein defining a wire path between two connection means further comprises selecting a location on each of the connection means for securing each end of the at least one wire support to the tray.34. The method of claim 25, wherein the wire path comprises various straight, angled, contoured, bent, or curved portions.

35. The method of claim 25, wherein the wire is comprised of one or more materials selected from the group consisting of one or more metals, steel, stainless steel, galvanized steel, copper, aluminum, one or more polymers, and any other materials suitable for sterilization.

36. The method of claim 25, wherein the at least one wire support has a cross-sectional thickness of about 0.05 to about 0.3 inches.
